(19) United States
(12) Patent Application Publication
(10) Pub. No.: US 2008/0194885 A1
(43) Pub. Date: Aug. 14, 2008

(54) Amine and membrane separation treament of liquid hydrocarbon media

(75) Inventors: Barry Abolmaali, San Diego, CA (US); Tiffany N. Morris, League City, TX (US); Norman Wise, Spring, TX (US); Kenneth M. Sulik, Lumberton, TX (US); Scott E. Lehrer, The Woodlands, TX (US)

Correspondence Address: WEGMAN, HESSLER & VANDERBURG, 6055 ROCKSIDE WOODS BOULEVARD, SUITE 200, CLEVELAND, OH, 44131, US

(73) Assignee: General Electric Company, Schenectady, NY (US)

(21) Appl. No.: 11/821,710

(22) Filed: Jun. 25, 2007

Related U.S. Application Data
(63) Continuation of application No. 11/215,399, filed on Aug. 30, 2005, now abandoned.

(51) Int. Cl. C07C 31/18

(52) U.S. Cl. 568/852

(57) ABSTRACT

Carbonyl species contamination of liquid hydrocarbon media and corrosion of metal surfaces in contact with such media are inhibited. A high boiling point primary or secondary amine is added to the desired liquid hydrocarbon medium, and in one exemplary embodiment, the medium is then brought into contact with a separatory membrane such as a nanofiltration membrane. The permeate from the membrane is a highly purified hydrocarbon stream.

### CROSS-REFERENCE TO RELATED APPLICATION

This application is a continuation of application Ser. No. 11/215,399 filed Aug. 30, 2005.

### FIELD OF INVENTION

The present invention pertains to a method to inhibit carbonyl species contamination of liquid hydrocarbon media and corrosion of metal surfaces that are in contact with such media. The method consists of a chemical treatment step with or without the use of a subsequent physical treatment step. The physical treatment step consists of contacting the chemically treated liquid hydrocarbon media with a semipermeable membrane.

### BACKGROUND OF THE INVENTION

Liquid hydrocarbon media such as those present in the petrochemical industry are often subject to contamination by the presence of carbonyl compounds therein. For example, carbon dioxide in such hydrocarbon process streams forms carbonic acid. This acid and other organic acids that are present can cause acid corrosion of metallurgy in contact with the process stream. Esters present in such streams can hydrolyze to acids. Further, aldehydes and other impurities in the liquid hydrocarbon stream or product can exceed required impurity levels and, if not separated from the process stream, result in product that does not meet purity requirements or end use specifications.

These problems are encountered for example in petrochemical processes adapted to form ethylene glycols. Ethylene glycols such as monoethylene glycol, diethylene glycol, triethylene glycol, etc., are important products and intermediates that are used in a variety of applications. For example, these products are useful in the preparation of textile fibers, antifreeze agents, hydraulic fluids, heat transfer agents, humectants and adhesives. Ethers of ethylene glycol are useful as solvents and chemical intermediates, particularly in the protective coatings industry.

In the preparation of polyester textile fibers, ethylene glycol is reacted with terephthalic acid to form the desired polymer. The ethylene glycol used in this process must be of the highest purity in order to form high quality polymer. One way of measuring the purity of the ethylene glycol is to subject it to a UV light transmittance test wherein excessive impurities results in lower than desired transmittance. Carbonyl species contamination of the ethylene glycol results in lower UV transmittance and may cause problems with regard to meeting desired UV and color specifications.

Ethylene glycols (e.g., monoethylene glycol, diethylene glycol, triethylene glycol, and tetraethylene glycol) may be prepared via several well known methods. In one method reported in U.S. Pat. No. 5,034,134, a two-stage reaction system is disclosed comprising a first step in which ethylene is oxidized over a suitable catalyst to form ethylene oxide. The so produced ethylene oxide is then reacted with water in a variety of stripping and reaction steps to ultimately form the desired ethylene glycols. The glycol stream containing water and undesirable carbonyl contaminants is subjected to one or more distillation steps to separate and purify the desired product.

### SUMMARY OF THE INVENTION

In accordance with the invention, carbonyl species contamination of liquid hydrocarbon process streams is decreased by addition of a high boiling amine or by using a two-step approach with amines in combination with a physical separation technique that uses membranes. The amine is chosen from high boiling primary and secondary amines and will inhibit acid based corrosion of system metallurgy and should exhibit thermal stability so that it will not volatilize during the heat processing steps that are employed so that it will therefore stay with the bottom stream in these processes.

The carbonyl based organic and inorganic contaminants, as mentioned above, react with the amine and then are removed when the hydrocarbon medium is contacted by a separatory membrane such as in one embodiment, a nanofiltration membrane. Although applicants are not to be bound to any theory of operation of the invention, it is thought that reaction of the amine with the impurities increases the size of the contaminates, thereby increasing the separation efficacy (i.e., reaction rate) of the separating membrane.

### DETAILED DESCRIPTION OF EXEMPLARY EMBODIMENTS

Although the invention will be primarily described in connection with its use in ethylene glycol production and purification processes, it is noteworthy that the invention is also applicable to other hydrocarbonaceous media such as those encountered in a variety of petrochemical processes such as olefinic or napthenic process streams, aromatic hydrocarbons and their derivatives, ethylene dichloride, and other processes. All of these are within the ambit of the phrase hydrocarbonaceous or hydrocarbon medium as used throughout the specification and claims. As is apparent to the artisan, significant amounts of water may also be present in such media.

Primary or secondary amines are added to the desired liquid hydrocarbonaceous medium in an amount of about 0.1-100 moles per mole of carbonyl function molecules present. Preferably, the treatment range is from about 0.5-10 moles of amine per mole of carbonyl functional molecules present. The amines should be chosen to have a sufficiently high enough boiling point to remain with the desired product during heat treating and purification processes such as distillation and fractionation.

In an ethylene glycol hydrocarbon stream including aqueous components, the amine should have a boiling point of about 200° C. or greater, preferably 300° C. or greater since the ethylene glycol stream is usually subjected to such temperatures during heat processing and purification. The glycol/water streams may, for example, be present anywhere within an ethylene oxide or ethylene glycol production or purification process.

In general, the amines that can be employed in accordance with the invention are characterized by the formula described in (I) or (II) below or a combination of (I) and (II).

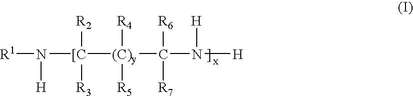

wherein R¹ is H, alkyl, cycloalkyl, or aryl; y is an integer from 0 to 9; x is an integer of from 1-10; and R¹-R⁶ are independently chosen from H, C₁-C₁₈ alkyl or C₁-C₁₈ alkyl substituted with hydroxyl, aryl, cycloalkyl, alkoxy, and amino groups.

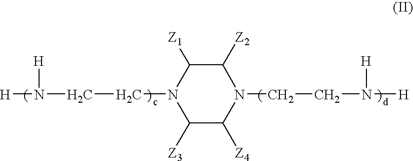

wherein c and d are independently chosen integers of from 0 to 3; Z₁, Z₂, Z₃, and Z₄ are independently chosen from H, OH, amino, C₁-C₁₂ alkyl, a hydroxyalkyl or aminoalkyl moiety of C₁-C₁₂ carbon atoms or aryl, preferably Z₁, Z₂, Z₃, and Z₄ are all H.

Preferred for use are the polyethylene polyamines having the formula

NH₂(CH₂CH₂NH)ₑH

wherein e is 2 or greater, preferably 3 to 10. Mixtures of these polyethylene polyamines may also be used. Present data suggests that tetraethylene pentamine is presently preferred with triethylenetetramine and pentaethylenehexamine also being exemplary.

In one embodiment of the invention, the liquid hydrocarbon medium that has been chemically treated as per above is contacted with a semipermeable membrane such as a nanofiltration membrane. Preferably, the pore size of the membranes is such that permeate molecules will have molecular weights of 300 Daltons or less, preferably 150 Daltons. The pore sizes are on the order of about 0.5-1.5 nm, preferably about 1.0 nm. The permeate, which is the material passing through the membrane, will have a lower concentration of carbonyl based impurities as compared to the impermeate or retentate stream which is the material that does not pass through the membrane. In those situations in which the combined chemical/physical separation steps of the invention are employed in an ethylene glycol process stream, the membrane separator will allow substantially all of the glycols to pass through the membrane while rejecting or inhibiting the chemically treated UV absorbers and/or other impurity components from doing so. This provides a high purity permeate with reduced UV absorbers and impurities therein. The permeate will consist primarily of water and glycols. The retentate (reject) stream will consist of the chemically treated UV absorbers and/or other impurity components, and any excess unreacted amine.

The chemical pretreatment not only reduces the amount of impurities, but also enhances the ability of the semi-permeable membrane to separate the impurities from the glycols and water at a substantially lower pressure (200-300 psig) than traditional semi-permeable membranes used to effect this separation. It should be understood that the rejection of the impurity components would be approximately 50% lower in the absence of chemical pretreatment prior to the physical separation step. Although applicants are not bound to any theory of operation of the invention, it is thought that reaction of the amine with the impurities increases the size of the contaminates, thereby decreasing the separation efficiency of the semi-permeable membrane.

One family of exemplary membrane separators that may be used in the invention is the D-Series of nanofiltration membranes available from GE. This is a spirally wound multilayer membrane in cylindrical form. Typically, these membranes operate at low feed pressures on the order of about 70-400 psig. The temperature of the feed is maintained at from about 0-100° C. Other exemplary membranes and operating conditions therefore are reported in U.S. Pat. No. 5,034,134 incorporated by reference herein.

The invention will be further described in conjunction with the following examples which should be viewed as being illustrative of exemplary embodiments and should not be construed to limit the invention.

### EXAMPLES

In order to assess the efficacy of the treatment compounds in reducing carbonyl species contamination in a liquid hydrocarbon medium, glycol process aldehyde scavenging tests were conducted. A feedstock comprising ethylene glycol/H₂O (40/10 v/v) was provided with aldehyde present in the medium in the amount indicated below. Tetraethylenepentamine/ethylene glycol candidate treatments were provided at 10% w/w.

Graduated cylinder vials were prepared with the liquid hydrocarbon medium and, where applicable, candidate treatment present. The vials were heated at 90° C. for 60 minutes. Following this reaction period, acetaldehyde concentration in the vapor phase was determined by gas chromatography. Results are as shown in Table I.

TABLE I

| ppm tetraethylenepentamine | ppm acetaldehyde |
|---|---|
| 0 | 249 |
| 537 | 120 |
| 1075 | 68 |
| 1612 | 50 |
| 2150 | 37 |

In accordance with the patent statutes, the best mode of practicing the invention has been set forth. However, it will be apparent to those skilled in the art that many other modifications can be made without departing from the invention herein disclosed and described.

What is claimed is:

1. A method for separating ethylene glycols from at least one carbonyl species impurity selected from the group consisting of carbon dioxide, carbonic acid, aldehydes and esters, said glycols and said impurity being dissolved or dispersed in a liquid medium containing said impurity and said ethylene glycols, said method comprising adding to said medium 0.1-100 moles per mole of said carbonyl species impurity of a secondary or tertiary amine having a boiling point of about 200° C. or greater to react with said impurity to form a reaction product in a treated medium, further comprising contacting said treated medium with a surface of a semi-permeable separatory membrane that exhibits selective permeation of said medium over that of said impurity and separating said treated medium into a permeate stream containing said liquid medium and an impermeate stream including said reaction product, said membrane adapted to pass as permeate molecules having a molecular weight of 300 Daltons or less.

2. Method as recited in claim 1 wherein said treated medium is heat treated at temperatures above about 200° C.

3. Method as recited in claim 2 wherein said amine comprises a polyamine having the formula:
NH₂(CH₂CH₂NH)ₑH
wherein e is 2 or greater.

4. Method as recited in claim 3 wherein said liquid medium comprises a member or members selected from ethylene glycol, diethylene glycol, and triethylene glycol and wherein said liquid medium further comprises water.

5. Method as recited in claim 4 wherein said amine comprises a member or members selected from the group consisting of triethylenetetraamine, tetraethylenepentamine, and pentaethylenehexamine.
